(19) United States
(12) Patent Application Publication
(10) Pub. No.: US 2007/0173848 A1
(43) Pub. Date: Jul. 26, 2007

(54) Bone jig

(75) Inventors: Iain Andrew Craig Lennox, Essex (GB); Russell Lloyd, Wiltshire (GB); Nick James Theophilius Metcalfe, Bath (GB)

Correspondence Address: HARNESS, DICKEY & PIERCE, P.L.C., P.O. BOX 828, BLOOMFIELD HILLS, MI, 48303, US

(73) Assignee: Biomet UK Limited, Bridgend (GB)

(21) Appl. No.: 10/545,927

(22) PCT Filed: Jul. 27, 2004
(86) PCT No.: PCT/GB04/03244

§ 371 Date: Oct. 16, 2006

(51) Int. Cl. A61F 5/00

(52) U.S. Cl. 606/87

(57) ABSTRACT

A jig includes a main body with at least one tool guide portion and a referencing member. The main body includes an adjusting member which acts between the main body and the referencing member to move the main body relative to the bone.

### FIELD

This invention relates to a bone jig and particularly, although not exclusively, relates to a jig for preparing the distal end of a femur.

### INTRODUCTION

If a knee joint becomes damaged or diseased, it is known to replace the entire knee joint with a prosthesis. There are a large variety of different knee prostheses, but the most common type consists of a femoral component attached to the distal end of the femur and a separate tibial component attached to the proximal end of the tibia. These components can articulate directly on one another or can be separated by a meniscal bearing component. Where possible, all of the knee ligaments are retained, although in practice it may be necessary to remove at least the posterior cruciate ligament. It may also be desirable for the tension in the knee ligaments after surgery to be balanced throughout their range of motion.

The most complex component of a total knee prosthesis is the femoral component, since it carries not only the condylar bearing surfaces, but also the patella bearing surface which extends along an anterior face of the distal femur. Conventional femoral components require resecting of the distal end surface of the femur and the anterior and posterior faces of the femur. They also usually require two chamfered cuts to be made at the distal end of the femur anteriorly and posteriorly. The anterior/posterior position of the cuts made in the femur are vital in order to restore proper functioning of the knee and balance to the ligaments. Conventional jigs for resecting the femur use as a reference an intramedullary rod with a set anterior/posterior position on the jig on the anterior/posterior axis. The correct positioning of the jig is vitally important to ensuring equal tension in the ligaments after surgery

### SUMMARY

A jig includes a main body having at least one tool guide portion, and a referencing member selectively attached to a bone. The main body includes an adjusting member which acts between the main body and the referencing member to move the main body relative to the bone.

According to various embodiments, the adjusting member comprises an adjusting screw adapted to engage a cooperating thread formed on the referencing member. The adjusting member may include one of a pinion and a worm. The adjusting member is captive in the main body. The referencing member may be pivotably connected to the main body such that it may pivot in a plane and the adjusting member moves the main body in a direction substantially perpendicular to the plane. The plane may define a varus/valgus plane and the direction may be an anterior/posterior direction.

A method of resecting a first bone, the first bone articulating with a second bone at a joint includes inserting a referencing member into the first bone with a longitudinal axis of an adjusting member aligned substantially perpendicular to an articulation axis of the joint, and with an underside of a main body engaging an end of the first bone. A spacer is inserted between a posterior surface of the main body and an end of the second bone. The adjusting member is adjusted until a desired tension is achieved in ligaments joining the first and second bone. The first bone is then resected.

Further areas of applicability of the present invention will become apparent from the detailed description provided hereinafter. It should be understood that the detailed description and specific examples, while indicating the embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein;

FIG. 1 is a perspective view of an exemplary cutting jig (or contour block) having a screw adjustable intramedullary rod;

FIG. 2 is a partially cut-away view of the jig of FIG. 1; and

FIG. 3 shows the jig of FIG. 1 attached to the distal end of an exemplary model of a human femur.

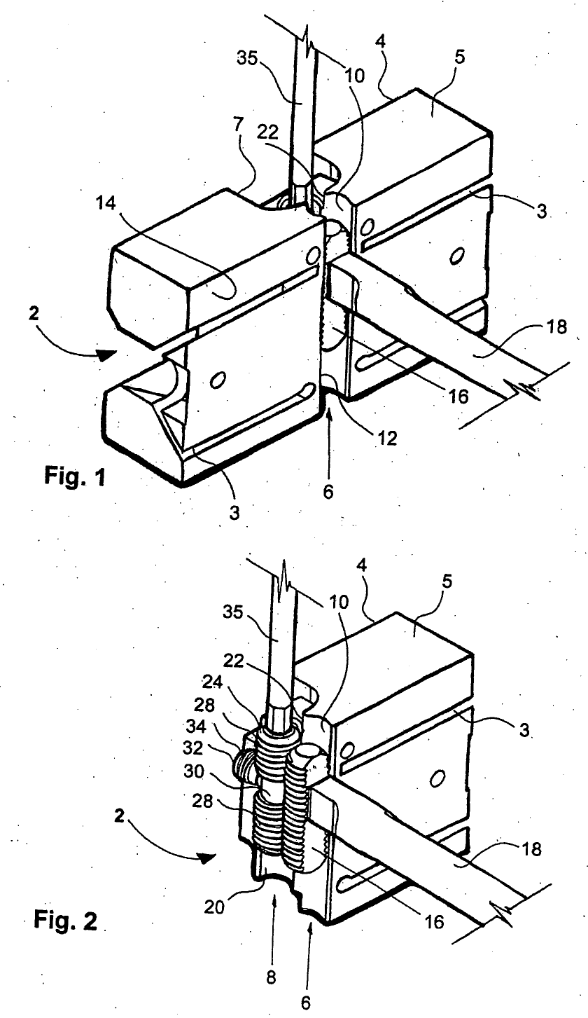

### DETAILED DESCRIPTION

The following description of the embodiments is merely exemplary in nature and is no way intended to limit the invention, its application, or uses. Moreover, while the description below is directed to a knee prosthesis, the invention and implant may be directed to any portion of the body.

Referring to the Figures, an exemplary jig 2 for resecting a distal femur comprises a main body or “block” 4 having a plurality of tool guides 3, for guiding the blade of a bone saw (not shown). Passing through the block 4 are a first opening 6, and a second opening 8 which may be parallel to the first opening 6. An anterior part 5 of the block 4 may be provided with an anterior pocket 7 which may be adapted to receive a calibrated stylus assembly 9 having a stylus point 11. The first opening 6 comprises a cylindrical bore 10 which may be integral with a slot 12 formed in a lower surface 14 of the block. Housed in the cylindrical bore 10 of the first opening 6 is a threaded pivot 16 which is connected to an intramedullary rod 18. The rod 18 passes through the slot 12 and projects downwardly from the lower surface 14 of the block. The second opening 8 comprises a second cylindrical bore 20 disposed adjacent the first cylindrical bore 10. The first opening 6 and second opening 8 overlap to form a second slot 22 which extends between the first opening 6 and second opening 8. An adjusting screw 24 is located in the first opening 6, and threaded portions 28, 28 of the adjusting screw 24 engage the threaded pivot 16 through the second slot 22.

Between the threaded portions 28, 28, the adjusting screw 24 is wasted to form an annular groove 30. A grub screw 32, which is threaded into an upper part 34 of the block 4, projects into the annular groove 30 and holds the adjusting screw 24 captive.

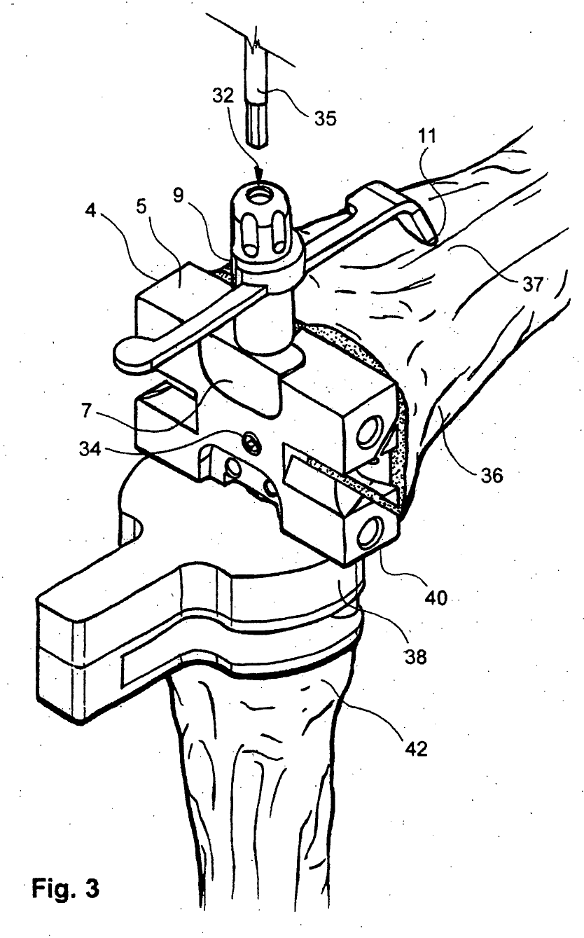

Referring particularly to FIG. 3, the stylus assembly 9 may be provided with a central bore 33 which provides access for an Allen key 35 to be inserted into the adjusting screw 24.

In a surgical procedure to implant a total knee prosthesis, the proximal tibia 42 can be resected using a standard technique, the distal femur 36 can be cut off, and the medullary canal (not shown) which extends approximately along the longitudinal axis of the femur 36, can be is exposed. The intramedullary rod 18 of the jig 2 can be inserted into the medullary canal such that the lower surface 14 of the block 4 contacts the resected upper surface of the distal femur 36. As the intramedullary rod 18 can pivot relative to the block 4, the lower surface 14 of the block can be brought into contact with the distal femur, whatever varus/valgus distal resection has been performed.

The stylus assembly 9 can be inserted into the anterior pocket 7 of the block 4, and the block 4 rotated about the longitudinal axis of the femur 36, until the stylus point 11 of the stylus assembly 9 is in contact with the anterior femoral cortex 37 of the femur 36. The stylus assembly 9 can be kept aligned with the anterior femoral cortex 37 throughout the anterior/posterior adjustment of the block 4 to avoid the possibility of notching the distal femur when an anterior cut is made.

With the knee at 90 degrees of flexion, a spacer 38 (or tensor device—now shown) can be inserted between a posterior surface 40 of the block 4 and the proximal end of the tibia 42. The thickness of the spacer 38 can be determined beforehand, with the leg in extension. An appropriate spacer 38 is selected such that it is closely received in the gap between the resected proximal tibia 42 and resected distal femur 36 in extension, when the required tension in the ligaments has been achieved and when the ligament tensions are balanced.

In order to accommodate the spacer 38 between the posterior surface 40 of the block 4 and the proximal tibia 42, the block 4 can be moved in an anterior/posterior direction relative to the distal femur 36 by adjusting the adjusting screw 24. As best appreciated from FIG. 2, the adjusting screw 24 can be captive in the block 4 and the intramedullary rod 18 can be fixed in the medullary canal of the femur 36. Consequently, as the adjusting screw 24 is rotated, it pulls itself along the threaded pivot 16, causing the block 4 to be moved in an anterior/posterior direction relative to the intramedullary rod 18 and distal femur 36.

The block 4 should be adjusted until it just seats flush against the spacer 38, thus providing the correct amount of external rotation of the block 4, according to the collateral ligament tension and the resected bone surfaces.

This allows the soft tissues to experience the same degree of tension as was present with the spacer 38 in extension, and assists in achieving correct rotational balance. If it is not possible to position the chosen block 4 adequately using translation of the block 4, the block 4 can be replaced with a block of a different size.

It should be noted that to avoid internal or incorrect rotation of the femur, it is important to address any soft tissue contractures prior to completing the positioning of the block 4. Marking Whitesides Line in the sulcus of the femur 36 can assist the visualisation of proper femoral rotation.

Once desired stability and good balance of the block 4 has been achieved, it may be secured to the femur with bone nails or screws (not shown). Two cutting guides (not shown) are then clicked into position, so that anterior and posterior condyle cuts and chamfer cuts can be performed on the distal femur 36.

The adjusting screw 24 provides stepless adjustment, so that the desired gap between the posterior surface 40 of the block 4 and the proximal tibia 42 can be set accurately by reference to the spacer 38. If the block 4 is knocked or otherwise interfered with, it will not move in an anterior/posterior direction, since the adjusting screw 24 will not turn unless rotated positively by the Allen key 35.

Once adjusted in this way, the same quadrilateral gap, with similar ligament tensions, is achieved in flexion, to that in extension, and the collateral ligaments are in equilibrium (thereby ensuring that the correct amount of external rotation has been introduced).

While the invention has been described in the specification and illustrated in the drawings with reference to various embodiments, it will be understood by those skilled in the art that various changes may be made and equivalents may be substituted for elements thereof without departing from the scope of the invention as defined in the claims. Furthermore, the mixing and matching of features, elements and/or functions between various embodiments is expressly contemplated herein so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the teachings of the invention without departing from the essential scope thereof. Therefore, it is intended that the invention not be limited to the particular embodiment illustrated by the drawings and described in the specification as the best mode presently contemplated for carrying out this invention, but that the invention will include any embodiments falling within the foregoing description and the appended claims.

What is claimed is:

1. A jig comprising:
a main body having at least one tool guide portion;
a referencing member selectively attached to a bone; and
wherein the main body includes an adjusting member which acts between the main body and the referencing member to move the main body relative to the bone.

2. The jig of claim 1 wherein the adjusting member comprises an adjusting screw adopted to engage cooperating thread formed on the referencing member.

3. The jig of claim 2 wherein the adjusting screw comprises one of a pinion and a worm.

4. The jig of claim 2 wherein the cooperating threads formed on the referencing member comprises a rack.

5. The jig of claim 1 wherein the adjusting member is captive in the main body.

6. The jig of claim 1 wherein the adjusting member provides stepless translation of the main body relative to the referencing member.

7. The jig of claim 1 wherein the referencing member is pivotably connected to the main body, so that it can pivot in a plane and the adjusting member moves the main body in a direction substantially perpendicular to the plane.

8. The jig of claim 7 wherein the plane defines a varus/valgus plane and direction is an anterior/posterior direction.

9. The jig of claim 1 wherein the tool guide portion comprises a slot for guiding a blade of a resecting device.

10. The jig of claim 1 wherein the tool guide portion is adapted to receive a removable tool guide.

11. The jig of claim 1 further comprising an alignment device attached to the main body which is adapted to reference the jig with an anterior cortex of a distal femur.

12. The jig of claim 1 wherein the adjusting member includes an overload device adapted to prevent the adjusting member from applying greater than a preset force to a joint.

13. The jig of claim 12 wherein the overload device is set to slip at a preset tightening torque of the adjusting member.

14. The jig of claim 1 wherein indicating means is provided to indicate at least one of an amount of adjustment and a force applied by the adjusting member.

15. The jig of claim 1 wherein the main body includes at least one drill guide.

16. The jig of claim 1 wherein the referencing member comprises an intramedullary rod.

17. The jig of claim 1 wherein the adjusting member engages at least one of the main body referencing members directly.

18. A method of resecting a first bone, the first bone articulating with a second bone at a joint comprising:
inserting a referencing member onto the first bone with longitudinal axis of an adjusting member aligned substantially perpendicular to an articulation axis of the joint, and with an underside of a main body engaging an end of the first bone;
inserting a spacer between a posterior surface of the main body and an end of the second bone;
adjusting the adjusting member until a desired tension is achieved in ligaments joining the first and second bone; and
resecting the first bone.

19. The A method as claimed in claim 18, further comprising, before the step (d), the step of fixing the jig to the first bone, in which the adjusting member comprises an adjusting screw adapted to rotate the main body relative to the referencing member and thereby adjust tension in the ligaments joining the first and second bones.

20. (canceled)

21. A jig comprising:
a main body having a tool guide portion;
a referencing member having a first series of threads;
an adjusting screw having a second series of threads threadably engaged with the first series of threads; and
wherein the adjusting screw advances the main body relative to the bone upon threadable translation between the first and second series of threads.
